(19) United States
(12) Patent Application Publication
(10) Pub. No.: US 2003/0078466 A1
(43) Pub. Date: Apr. 24, 2003

(54) Method for treating muscle in wall and apparatus for same

(76) Inventors: David E. Silverman, Palo Alto, CA (US); Alan Stein, Moss Beach, CA (US)

Correspondence Address: Dorsey & Whitney LLP, Intellectual Property Department, Four Embarcadero Center, Suite 3400, San Francisco, CA, 94111-4187, US

(21) Appl. No.: 10/233,044

(22) Filed: Aug. 30, 2002

Related U.S. Application Data
(60) Provisional application No. 60/316,578, filed on Aug. 31, 2001.

(51) Int. Cl. A61F 2/00

(52) U.S. Cl. 600/29

(57) ABSTRACT

A method for treating a body of a mammal having a natural body passageway formed by a wall having a muscle layer. The method includes the step of forming at least one tightening in the muscle layer to reduce the distensibility of the muscle layer.

### CROSS REFERENCE TO RELATED APPLICATION

This application claims priority to U.S. provisional patent application Serial No. 60/316,578 filed Aug. 31, 2001, the entire content of which is incorporated herein by this reference.

### FIELD OF THE INVENTION

The present invention relates generally to the treatment of muscle in a wall of a mammalian body and more particularly to the introduction of a material into the muscle in a wall to treat the wall.

### BACKGROUND OF THE INVENTION

Methods and apparatus have been provided for introducing materials into a wall of a mammalian body to treat the wall. See in this regard U.S. Pat. Nos. 6,238,335 and 6,251,063.

### SUMMARY OF THE INVENTION

One method of the invention provides for treating a body of a mammal having a natural body passageway formed by a wall having a muscle layer. The method includes the step of forming at least one tightening in the muscle layer to reduce the distensibility of the muscle layer.

Another method of the invention provides for treating muscle in a body of a mammal having an esophagus extending through a lower esophageal sphincter to a stomach and formed by a wall having a muscle layer. The method includes the step of forming at least one tightening in the wall in the vicinity of the lower esophageal sphincter to inhibit opening of esophagus at the lower esophageal sphincter.

Another method of the invention provides for increasing the competency of a sphincter having tone in a gastrointestinal tract having a chamber extending to the sphincter. The chamber has a pressure therein and is defined by a wall having a muscle layer. The method includes the step of creating a tightening in the muscle layer in the vicinity of the sphincter. With increased pressure in the chamber the tightening causes the sphincter to retain tone thereby lessening incontinence of the chamber.

In one embodiment of the invention, an apparatus is provided for treating muscle in a body of a mammal having an esophagus extending through a lower esophageal sphincter to a stomach and formed by a wall having a muscle layer. The apparatus includes an elongate probe member having proximal and distal extremities. A stylet is carried by the elongate probe member and is extendable from the distal extremity. A temperature sensor is carried by the stylet for measuring the temperature of the muscle during treatment.

In another embodiment of the invention, an apparatus is provided for treating muscle in a body of a mammal having an esophagus extending through a lower esophageal sphincter to a stomach and formed by a wall having a muscle layer. The apparatus includes an elongate probe member having proximal and distal extremities. A stylet is carried by the elongate probe member and is extendable from the distal extremity. An oxygen sensor is carried by the stylet for measuring the oxygen concentration of the muscle during treatment.

### BRIEF DESCRIPTION OF THE DRAWINGS

FIG. 1 is a perspective view of an apparatus for treating muscle of the present invention.

FIG. 2 is a schematic view of the apparatus for treating muscle of FIG. 1 practicing the method of the present invention.

FIG. 3 is an enlarged view of the lower esophageal sphincter of FIG. 2.

### DESCRIPTION OF THE INVENTION

The methods hereof and related apparatus can be used in any organ in a body of a mammal. The methods and related apparatus are particularly suited for use in human bodies, preferably natural body cavities accessible by natural body openings and more preferably in the lower esophageal sphincter, the stomach and the pylorus of the gastrointestinal tract. The method is particularly suited to treating the muscle layer of the gastrointestinal tract in the vicinity of sphincters or other similar valve-like mechanisms.

As used herein, the term “sclerosing agent” can be any material capable of causing or assisting to initiate fibrosis or a fibrotic reaction in tissue of a body. In one preferred embodiment, any suitable fluid from which a fibrotic reaction can be initiated when the fluid, separately or together with another fluid, is introduced into the body can be utilized for causing and forming fibrosis and secondary sclerosis in a body. Aqueous or nonaqueous solutions are suitable fluids. Preferred nonaqueous solutions include any of the organic solvents that, when in contact with a liquid or an aqueous solution, such as that contained in tissue, cause an exothermic reaction, liberating heat therein.

Suitable sclerosing agents or materials include any material capable of being delivered through a needle (preferably a small-gauge needle), solutions, suspensions, slurries, biodegradable or nonbiodegradable materials and two part or other mixtures. Exemplary sclerosing or fibrosing materials include dimethyl sulfoxide (hereinafter “DMSO”) and other known injectable materials which, when in contact with tissue at low concentrations or volumes cause a non-toxic reaction and at higher concentrations or in greater volumes liberate enough heat to cause a continuum of tissue change beginning with inflammation and progressing through necrosis, fibrosis and sclerosis. The amount of heat liberated is proportional to the volume of DMSO that comes in contact with the tissue.

Any of the devices disclosed or discussed in U.S. Pat. No. 6,238,335 issued May 29, 2001 and U.S. Pat. No. 6,251,063 issued Jun. 26, 2001 and in International Application No. PCT/US99/29427, the entire content of each of which is incorporated herein by this reference, can be utilized with the method described herein. In general, such a suitable device has a probe member with a needle assembly slidably disposed therein. The needle assembly preferably includes a sleeve slidably disposed on a needle. The distal extremity of the needle assembly is extendable and retractable relative to the distal extremity of the probe member and the distal extremity of the needle is extendable and retractable relative to the distal extremity of the sleeve. A reservoir of the injectable material is coupled to the proximal extremity of the device. In addition thereto, such devices can be adapted to include a servomechanism which monitors any one or combination of the temperature, oxygen concentration or partial pressure in the tissue during treatment and, by feedback, controls the introduction of the sclerosing material.

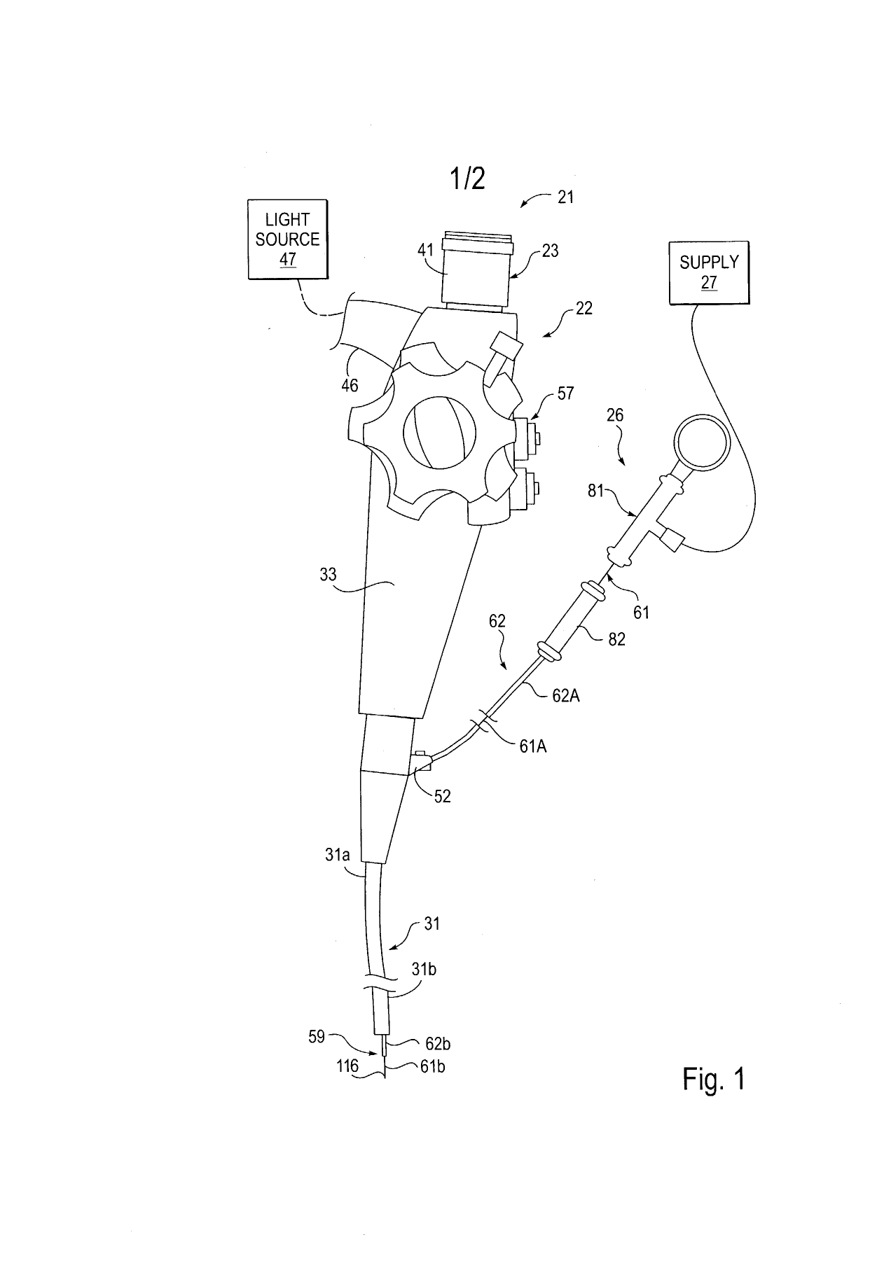

One embodiment of a device utilized with the method of the present invention is shown in FIGS. 1-3. Apparatus or medical device 21 shown therein includes a probe member or probe 22 having an optical viewing device 23. A stylet or needle assembly 26 is slidably carried by probe 22. Treatment device 21 further includes a supply assembly or supply 27 mounted to the proximal end portion of needle assembly 26.

A conventional or other suitable gastroscope or endoscope can be used for probe 22. Probe 22 includes a flexible elongate tubular member or insertion tube 31 having proximal and distal extremities 31a and 31b and a distal face 32. Insertion tube 31 has been sectioned in FIG. 1 so that only a portion of proximal extremity 31a and distal extremity 31b are shown. A handle means or assembly is coupled to proximal extremity 31a of the insertion tube 31 and includes a conventional handle 33. The tubular insertion tube 31 is provided with a plurality of bores or passageways (not shown) extending from proximal extremity 31a to distal extremity 31b.

Optical viewing device 23 is formed integral with conventional probe 22 and has an optical element or objective lens (not shown) carried by one of the passageways of insertion tube 31. The lens has a field of view which permits the operator to view forwardly of insertion tube distal extremity 31b. Optical viewing device 37 further includes an eye piece 41 mounted on the proximal end of handle 33. A connection cable 46, a portion of which is shown in FIG. 1, extends from handle 33 to a conventional light source 47. At least one light guide (not shown) extends through insertion tube 31 and cable 46 for providing illumination forwardly of insertion tube 31.

A working passageway or channel (not shown) is further provided in insertion tube 31 and extends to a side port 52 formed in handle 33. Insertion tube 31 is flexible so as to facilitate its insertion and advancement through a body and is provided with a bendable distal end for selectively directing its distal face in a desired direction. A plurality of finger operable controls 57 are provided on handle 33 for, among other things, operating the bendable distal end of insertion tube 31 and the supply and removal of fluids through the insertion tube 31.

Needle assembly 26 can be of any conventional type such as a modified sclerotherapy needle. The needle assembly 26 includes a stylet 59 having a needle member or needle 61 having a proximal end portion 61a and a distal end portion 61b and an optional sleeve member or sleeve 62 having a proximal end portion or extremity 62a and a distal end portion or extremity 62b. Sleeve or elongate tubular member 62 is made from any suitable material such as flexible plastic or metal and has a lumen extending longitudinally therethrough for receiving the needle 61. The sleeve 62 and the needle 61 are slidable relative to each other in a longitudinal direction. In this regard, tubular needle 61 is slidably disposed in sleeve 62 and movable from a retracted position in which the tubular needle is recessed within distal end portion 62b of sleeve to an extended position in which the needle 61 projects distally of the sleeve 62. The needle has a passageway or lumen (not shown) therein for permitting the passage of a fluid through the needle.

A fluid connector 81 is secured or coupled to proximal end portion 61a of needle 61 and a gripping member or grip 82 is secured to the proximal end portion 62a of the sleeve 62 (see FIG. 1). Fluid connector 81 includes at least one luer fitting portion 83, or any other suitable fitting portion, which communicates with the passage in needle 61. Supply 27 can be of any suitable type such as a syringe 83 operated by a servo-mechanism 84 for containing the sclerosing agent or material of the present invention.

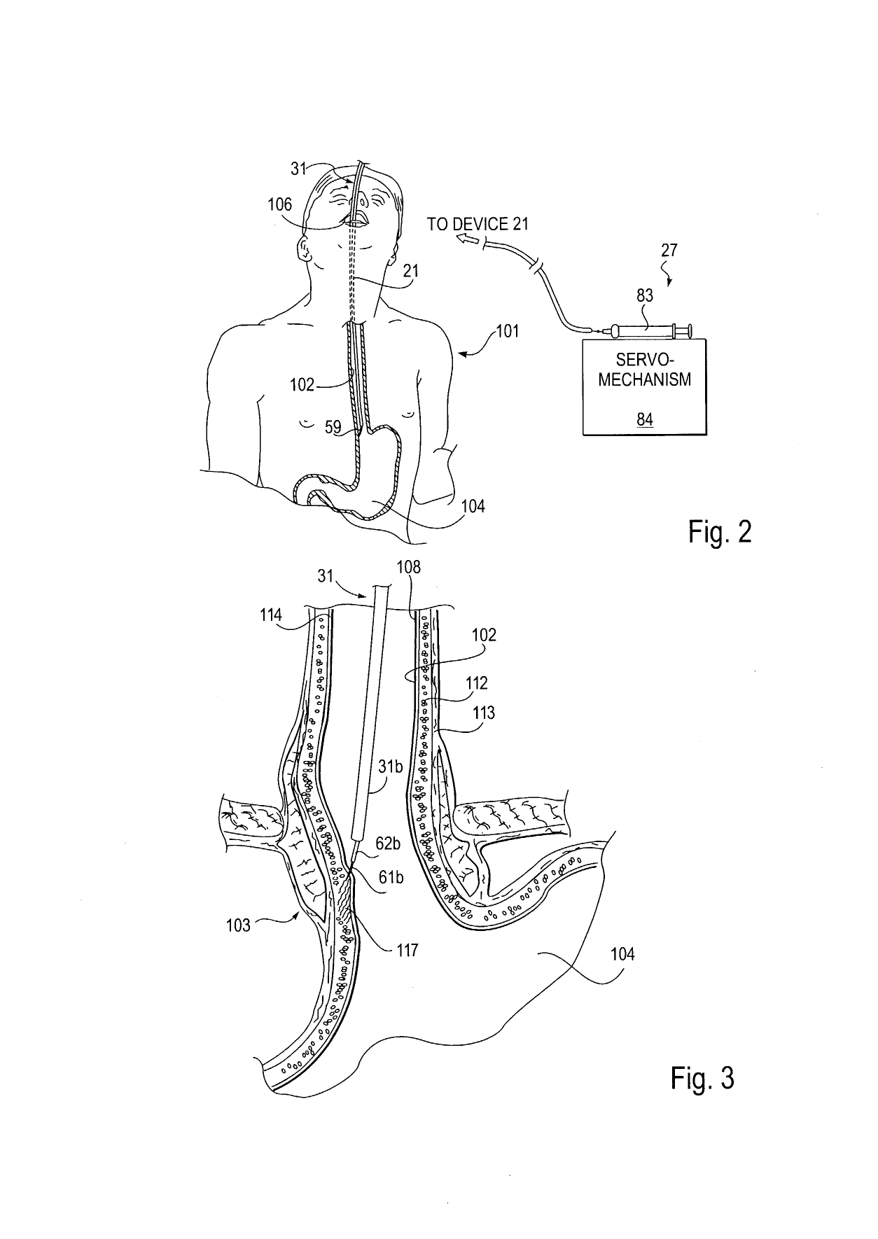

The method of the present invention is for use in a natural body cavity such as the gastrointestinal tract in a body of a mammal. A portion of a human body 101 is shown in FIGS. 2 and 3 and has an internal cavity in the form of the passage of the esophagus 102 extending through a lower esophageal sphincter 103 to a stomach 104. Such cavity is accessible by a natural body opening in the form of mouth 106 and is defined by wall 107. Esophagus 102 and stomach 104 form part of the gastrointestinal tract of body 101 that extends from mouth 106 to an anus (not shown).

Esophageal mucosa 108 serves as the inner layer of the intraluminal wall 107 in esophagus 102. The wall 107 has a muscle layer comprising a layer of circular muscle 112 extending beneath mucosa layer 108 and a layer of longitudinal muscle 113 beneath circular muscle 112. Muscle layers 112 and 113 extend around esophagus 102 and stomach 104. Wall 107 further includes a submucosal layer or submucosa 114 extending between mucosa 108 and muscle layers 112 and 113. A submucosal space, that is a potential space, can be created between submucosa 114 and circular muscle layer 112 by the separation of layer 108 from muscle layer 112. In addition, as with any muscle, wall 107 includes an intramuscular potential space, that is a space which can be created intramuscularly by distension and separation of muscle fibers within a single muscle. Wall 107 has a depth or thickness which includes at least mucosal layer 108, submucosal layer 114, circular muscle layer 112 and longitudinal muscle layer 113.

The lower esophageal sphincter or LES 103 shown in FIG. 3 is a one-way sphincter that serves as the anatomical junction between the esophagus 102 and stomach 104. The device 21 is placed within the esophagus and stomach of a patient, as shown in FIGS. 2 and 3. A sensor 116, such as a thermistor or other temperature sensor and/or an oxygen electrode or other type of oxygen sensor, is optionally carried by the distal extremity of the stylet 59 for measuring the temperature and partial pressure of oxygen in the muscle during treatment. Sensor 116 is shown in FIG. 1 as being carried by needle distal extremity 61b. The thermistor and/or electrode is coupled by one or more wires carried within a lumen of the stylet, such as the lumen of the needle, to servomechanism 84 which registers the temperature and/or oxygen partial pressure and is configured to adjust the rate and total volume of sclerosing agent introduced during treatment in response thereto. Such a temperature sensor is used, for example, to measure the temperature of the tissue acted upon by the injectable material. For example, when DMSO comes into contact with tissue heat is generated from the resulting exothermic reaction. If the measured temperature is not sufficient to create a fibrosis or necrosis in the tissue, more of the injectable material can be added to the tissue. Such an oxygen sensor detects the oxygen concentration in the tissue. The oxygen concentration in healthy tissue is much higher than the oxygen concentration in tissue that has been necrosed or sclerosed. Although sensor 116 is shown as being carried by needle 61, it should be appreciated that the sensor 116 can also be carried by the distal end of the sleeve 62.

In the illustrated embodiment, the invention is directed to treating gastroesophageal reflux disease. In the first aspect of the method of the invention, the sclerosing material is injected into one or more locations in the wall 107 of the esophagus 102, preferably at or in the vicinity of the muscle of the lower esophageal sphincter 103, to cause fibrosis and secondary sclerosis thereby thickening and tightening the LES. This creates a stenosis, tightening, fibrotic reaction or fibrosis in the LES and reestablishes or increases the tone and competency of the lower esophageal sphincter. Initially, if the solution is not injected at a basal rate which is great enough to cause a sufficient exothermic reaction when the solvent contacts targeted tissue, the rate and/or total volume of solution introduced can be increased, either manually or by means of a servo-mechanism or similar device. Similarly, if the rate of injection is not adequate to cause a sufficient drop in the partial pressure of oxygen in the targeted tissue (indicating injury and/or necrosis of at least a portion of the targeted tissue), the rate and/or volume of solution introduced can be augmented. An exemplary necrosis or stenosis 117 ultimately caused by adequate rates and volumes of injection is shown in FIG. 3 in circular muscle layer 112.

The injectable material can be injected into any layer of the wall, such as the mucosa 108 or the submucosa 114, although it is preferred that the sclerosing material be deposited into the muscle in the wall. Although the injectable agents do not reestablish an anatomic sphincter, if injected into the muscle layer they do decrease distensibility of the muscular wall by creating some level of long-term sclerosis or scar tissue, thereby forming a semi-permanent or permanent tightening in the muscular layer. The tightening decreases gastroesophageal reflux by permitting the augmented resting tone of the LES to protect against or overcome an increase in gastric pressure.

The location and resultant configuration of the stenoses 117 can vary. In this regard, the sclerosis can be caused to be circular or ring-like. Alternatively, the scleroses can be created with finger-like or lake-like extensions or, similar to the shape of the implants disclosed in U.S. Pat. No. 6,251,064 issued Jun. 26, 2001, the entire content of which is incorporated herein by this reference, to extend arcuately around at least a portion of the esophagus and possibly completely around the esophagus.

The number and configurations of fibrotic or sclerotic lesions 117 formed in the wall of the gastrointestinal tract can vary. Specific examples of lesion formations are similar to the implant configurations disclosed in U.S. Pat. No. 6,251,063 issued Jun. 26, 2001.

The approach and the access to the esophagus 102 for the method of the invention can be endoscopic, laproscopic or by open surgery. The introduction of the injectable material can be transmucosal, from the inside towards the out, or transserosal, from the outside in.

1. A method for treating a body of a mammal having a natural body passageway formed by a wall having a muscle layer comprising the step of forming at least one tightening in the muscle layer whereby the at least one tightening reduces the distensibility of the muscle layer.

2. The method of claim 1 wherein the forming step includes the step of introducing at least one sclerosing agent into the muscle layer to form the at least one tightening.

3. The method of claim 2 wherein the sclerosing agent includes DMSO.

4. A method for treating muscle in a body of a mammal having an esophagus extending through a lower esophageal sphincter to a stomach and formed by a wall having a muscle layer comprising the step of forming at least one tightening in the wall in the vicinity of the lower esophageal sphincter whereby the at least one tightening inhibits opening of esophagus at the lower esophageal sphincter.

5. The method claim 4 wherein the forming step includes the step of forming the at least one tightening in the muscle layer of the wall.

6. The method of claim 5 wherein the forming step includes the step of forming a plurality of circumferentially-disposed tightenings in the muscle layer of the wall.

7. The method of claim 4 wherein the forming step includes the step of introducing a fluid into the muscle layer of the wall and forming the at least one tightening from the fluid.

8. The method of claim 7 wherein the fluid includes at least one sclerosing agent and wherein the forming step includes the step of forming at least one tightening from the at least one sclerosing agent.

9. The method of claim 7 wherein the fluid includes at least one solvent and wherein the forming step includes the step of forming at least one tightening from the at least one solvent.

10. The method of claim 8 wherein the sclerosing agent includes dimethyl sulfoxide (DMSO) and wherein the forming step further includes the step of forming at least one tightening from the DMSO.

11. The method of claim 4 wherein the forming step includes the step of forming an arcuate tightening in the muscle layer which extends around at least a portion of the wall.

12. The method of claim 11 wherein the arcuate tightening is ring shaped.

13. A method for increasing the competency of a sphincter having tone in a gastrointestinal tract having a chamber extending to the sphincter, the chamber having a pressure therein and defined by a wall having a muscle layer comprising the step of creating a tightening in the muscle layer in the vicinity of the sphincter whereby with increased pressure in the chamber the tightening causes the sphincter to retain tone thereby lessening incontinence of the chamber.

14. The method of claim 13 wherein the creating step includes the step of injecting a solution into the muscle layer whereby at least one tightening is formed from the solution.

15. The method of claim 14 wherein the solution includes at least one organic solvent and the creating step further includes the step of forming a tightening from the organic solvent.

16. The method of claim 14 wherein the solution includes at least one sclerosing agent and wherein the creating step further includes the step of forming a tightening from the sclerosing agent.

17. The method of claim 16 wherein the agent is dimethyl sulfoxide and wherein the forming step further includes the step of eliciting a fibrotic reaction from the dimethyl sulfoxide.

18. The method of claim 16 further including the steps of eliciting an exothermic reaction during injection of the solution and monitoring the temperature in the muscle layer during injection.

19. The method of claim 18 further including the steps of controlling the rate and amount of injection of the solution based upon said monitoring of the temperature in the muscle layer.

20. The method of claim 19 further including the step of automating the controlling step.

21. The method of claim 16 further including the steps of monitoring the oxygen concentration in the muscle layer during injection of the solution and controlling the rate and amount of injection based upon the concentration.

22. The method of claim 16 further including the steps of monitoring a property of the muscle layer and controlling the amount of the sclerosing agent injected into the muscle layer based upon such property.

23. An apparatus for treating muscle in a body of a mammal having an esophagus extending through a lower esophageal sphincter to a stomach and formed by a wall having a muscle layer comprising an elongate probe member having proximal and distal extremities, a stylet carried by the elongate probe member and extendable from the distal extremity and a temperature sensor carried by the stylet for measuring the temperature of the muscle during treatment.

24. An apparatus for treating muscle in a body of a mammal having an esophagus extending through a lower esophageal sphincter to a stomach and formed by a wall having a muscle layer comprising an elongate probe member having proximal and distal extremities, a stylet carried by the elongate probe member and extendable from the distal extremity and an oxygen sensor carried by the stylet for measuring the oxygen concentration of the muscle during treatment.

25. The apparatus of claim 24 wherein the oxygen sensor is an oxygen electrode.
